(19) United States
(12) Patent Application Publication
(10) Pub. No.: US 2016/0199296 A1
(43) Pub. Date: Jul. 14, 2016

(54) NASAL FORMULATIONS OF BENZODIAZEPINE

(71) Applicant: CPEX PHARMACEUTICALS, INC., Wilmington (DE)

(72) Inventors: Nils Bergenhem, Guilford, CT (US); Carl Reppucci, North Andover, MA (US); Zhengmao Li, New Market, NH (US)

(21) Appl. No.: 15/079,576

(22) Filed: Mar. 24, 2016

Related U.S. Application Data
(63) Continuation of application No. 14/007,153, filed on Nov. 18, 2013.
(60) Provisional application No. 61/470,823, filed on Apr. 1, 2011.

(51) Int. Cl. A61K 9/00; A61K 31/365; A61K 31/5513

(52) U.S. Cl. CPC A61K 9/0043; A61K 31/5513; A61K 31/365

(57) ABSTRACT

The present invention provides pharmaceutical compositions for intranasal delivery of a benzodiazepine. The composition may contain a therapeutically effective amount of a benzodiazepine or a pharmaceutically acceptable salt thereof, and a permeation enhancer. The permeation enhancer may be propylene glycol or a Hsieh permeation enhancer. The present compositions have excellent bioavailability. After administration of the present compositions, therapeutically effective plasma levels of the benzodiazepine may be achieved rapidly. The present pharmaceutical composition may be used to treat a patient suffering from anxiety, epilepsy, insomnia, agitation, seizures, muscular disorders, alcohol dependence, and drug withdrawal.

### CROSS REFERENCE TO RELATED APPLICATION

This application claims priority to U.S. Provisional Application No. 61/470,823 filed on Apr. 1, 2011, which is incorporated herein by reference in its entirety.

### FIELD OF THE INVENTION

The present invention relates to benzodiazepine compositions. In particular, the present invention relates to compositions and methods for the delivery of benzodiazepines through the nasal mucosa of a mammal.

### BACKGROUND OF THE INVENTION

Benzodiazepines are a class of psychoactive drugs that act primarily on the central nervous system. Specifically, benzodiazepines enhance the effect of the neurotransmitter gamma-aminobutyric acid (GABA), resulting in sedative, hypnotic (sleep-inducing), anxiolytic (anti-anxiety), anticonvulsant, muscle relaxant and/or amnesic effects. Page et al. Integrated Pharmacology (2006) (3rd ed.). Published by C.V. Mosby. Thus, benzodiazepines are useful in treating conditions including anxiety, epilepsy, insomnia, agitation, seizures, muscular disorders, and alcohol dependence. These drugs can also be used as a premedication for medical or dental procedures. Olkkola et al., “Midazolam and other benzodiazepines”. Handbook Exp. Pharmacol. 2008, 182 (182): 335-60.

Most benzodiazepines are administered orally. They can also be given intravenously, intramuscularly or rectally. Royal Pharmaceutical Society of Great Britain (2009). British National Formulary (BNF 57). BMJ Group and RPS Publishing. However, oral administration does not allow therapeutic plasma levels of benzodiazepines to be achieved rapidly. The concentration of drugs that finally reach the central nervous system is also decreased by hepatic first-pass metabolism. Intravenous or intramuscular injections, as well as rectal administration, have numerous drawbacks such as discomfort to the patient, poor patient compliance, and the need for administration by trained technicians.

Intranasal administration offers a desired non-invasive alternative to deliver a benzodiazepine to the central nervous system effectively. A number of hydrophilic and lipophilic therapeutic agents have been shown to enter the brain directly from the nasal cavity via olfactory pathway. Illum, L., 2000. Transport of drugs from the nasal cavity to the central nervous system. Eur. J. Pharm. Sci. 11, 1-18. In addition, this route can provide rapid absorption of drugs into the blood circulation through the nasal vasculature. The pharmacokinetics can be similar to those achieved for intravenous administration. Hashizume et al. New therapeutic approach for brain tumors: intranasal delivery of telomerase inhibitor GRN163. Neuro-oncology 10: 112-120, 2008. Thorne et al. Delivery of insulin-like growth factor-1 to the rat brain and spinal cord along olfactory and trigeminal pathways following intranasal administration. Neuroscience 127: 481-496, 2004. Such rapid and effective drug delivery is particularly suitable for a prompt medication of patients in the acute and/or emergency circumstances, such as status epilepticus and other fever-induced seizures. For example, a seizing patient suffers from rigid muscles and uncontrollable movements, which can make oral or intravenous administration difficult, even dangerous. However, the nasal passageways remain easily accessible for drug delivery.

Despite its advantages, intranasal delivery of benzodiazepines has had limited success. Many benzodiazepines are substantially insoluble or only sparingly soluble in water; thus, non-aqueous solvents may be required to dissolve these agents. However, such solvents are frequently too irritating to sensitive mucosal tissues to be of clinical use. A further constraint concerning nasal delivery is that a small administration volume is required. For example, it is not generally recommended to administer more than about 0.1 ml to 0.2 ml per dose per nostril. Therefore, there exists a great need for suitable benzodiazepine compositions, in which, on one hand, the solubility of the benzodiazepine is good, and which, on the other hand, are non-irritating to the mucosa and have high bioavailability.

### SUMMARY

The present invention provides for a pharmaceutical composition for nasal administration comprising a therapeutically effective amount of a benzodiazepine or a pharmaceutically acceptable salt thereof, and a permeation enhancer. The relative bioavailability of the composition compared to a rectal formulation of benzodiazepine may range from about 80% to about 500%, from about 150% to about 400%, from about 250% to about 300%, from about 80% to about 200%, from about 100% to about 150%, or from about 100% to about 120%. The composition is substantially non-irritating to the nasal mucosa.

After administration of a single dose of the pharmaceutical composition, Tmax of plasma benzodiazepine may range from about 1 minute to about 10 minutes, about 5 minutes, or about 10 minutes; Cmax of plasma benzodiazepine may range from about 15 to about 800 ng/ml when plasma benzodiazepine is measured from about 0 to about 2 hours. Dose-normalized Cmax of plasma benzodiazepine may range from about 3 to about 60 ng/ml/mg. AUC_{0-5 min} of plasma benzodiazepine may range from about 50 to about 500 ng*min/ml. AUC_{0-10 min} of plasma benzodiazepine may range from about 150 to about 1,400 ng*min/ml.

The benzodiazepine may be diazepam, alprazolam, bentazepam, bromazepam, brotizolam, chlordiazepoxide, clobazam, clonazepam, clorazepam, clotiazepam, delorazepam, demoxazepam, estazolam, ethyl loflazepate, etizolam, fludiazepam, flumazenil, flunitrazepam, flurazepam, halazepam, ketozolam, lorazepam, loprazolam, lormetazepam, medazepam, mexazolam, midazepam, midazolam, nimetazepam, nitrazepam, nordazepam, oxazepam, pinazepam prazepam, quazepam, temazepam, tetrazepam, triazolam or mixtures thereof. The amount of the benzodiazepine may range from about 0.1% (w/w) to about 50% (w/w), from about 1% (w/w) to about 30% (w/w), or from about 1% (w/w) to about 10% (w/w) of the composition.

The permeation enhancer may be a hydroxyl group-containing compound, such as propylene glycol. The amount of propylene glycol may be greater than about 30% (w/w) of the composition, or greater than about 80% (w/w) of the composition.

The permeation enhancer may be a Hsieh enhancer having the following structure:

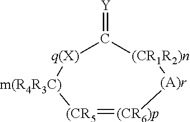

wherein X and Y are oxygen, sulfur or an imino group of the structure

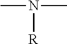

or ═N—R with the proviso that when Y is the imino group, X is an imino group, and when Y is sulfur, X is sulfur or an imino group, A is a group having the structure

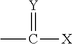

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11.

Non-limiting examples of the Hsieh enhancers include 3-methylcyclopentadecanone, 9-cycloheptadecen-1-one, cyclohexadecanone, cyclopentadecanone, oxacyclohexadecan-2-one and mixtures thereof. The amount of the Hsieh enhancer may range from about 0.001% (w/w) to about 40% (w/w) of the composition.

The composition may also contain a solvent such as ethanol, Cremophor EL, any forms of polyethoxylated castor oil, dipropylene glycol, dimethyl isosorbide, water, and mixtures thereof. The composition may contain a crystallization inhibitor. The composition can be administered by an intranasal spray device, an atomizer, a nebulizer, a metered dose inhaler (MDI), a dry powder inhaler (DPI), a pressurized dose inhaler, an insufflator, an intranasal inhaler, a nasal spray bottle, a unit dose container, a pump, a dropper, a squeeze bottle, or a bi-directional device. The composition may be administered to a patient suffering from anxiety, epilepsy, insomnia, agitation, seizures, muscular disorders, alcohol dependence, or drug withdrawal.

### BRIEF DESCRIPTION OF THE DRAWINGS

FIG. 1 depicts the dose—exposure relation of administering intranasal diazepam compositions according to the present invention. These intranasal compositions (2.5 mg, 4 mg and 10 mg doses) were compared to Diastat® (diazepam rectal gel; 4 mg dose) which was used as a positive control. The plasma diazepam concentration shown is dose-normalized.

FIG. 2 depicts the dose—exposure relation of administering intranasal diazepam compositions according to the present invention. These intranasal compositions (2 mg, 1 mg and 2 mg doses) were compared to Diastat® (diazepam rectal gel; 4 mg dose) which was used as a positive control. The plasma diazepam concentration shown is dose-normalized.

FIG. 3 depicts the dose—exposure relation of administering intranasal diazepam compositions according to the present invention. These intranasal compositions (2 mg, 1 mg and 2 mg doses) were compared to Diastat® (diazepam rectal gel; 4 mg dose) which was used as a positive control.

FIG. 4 depicts the dose—exposure relation of administering diazepam intravenously or rectally. Diastat® (diazepam rectal gel; 4 mg dose) were compared to intravenous diazepam (2 mg dose) which was used as a positive control. The plasma diazepam concentration shown is dose-normalized.

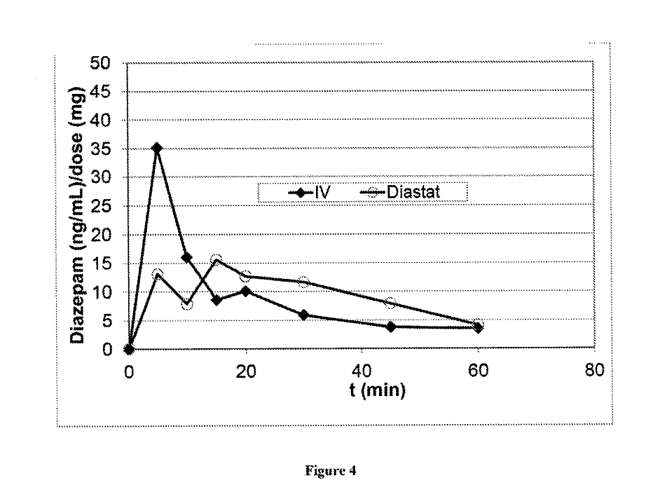

### DETAILED DESCRIPTION OF THE INVENTION

The present invention provides pharmaceutical compositions for intranasal delivery of a benzodiazepine. The intranasal compositions could provide much higher benzodiazepine bioavailability compared to a rectal formulation of benzodiazepine. Relative bioavailability of the present composition compared with a rectal formulation may range from about 80% to about 500%, from about 100% to about 500%, from about 120% to about 500%, from about 150% to about 400%, from about 200% to about 300%, from about 250% to about 300%, from about 80% to about 200%, from about 80% to about 150%, from about 80% to about 120%, from about 100% to about 200%, from about 100% to about 150%, or from about 100% to about 120%.

The present composition is substantially non-irritating to the nasal mucosa. After administration of the present compositions, therapeutically effective plasma levels of the benzodiazepine may be achieved rapidly, for example, in less than about 2 hours, less than about 1.5 hours, less than about 1 hour, less than about 45 minutes, less than about 30 minutes, less than about 15 minutes, less than about 10 minutes, or less than about 5 minutes. The present pharmaceutical composition may be used to treat a patient suffering from any disorder or condition that is amenable to treatment with an effective amount of at least one benzodiazepine. Non-limiting examples of such disorders or conditions include: anxiety, epilepsy, insomnia, agitation, seizures, muscular disorders, alcohol dependence, and drug withdrawal. In certain embodiments, the present composition contains a therapeutically effective amount of a benzodiazepine or a pharmaceutically acceptable salt thereof, and a Hsieh permeation enhancer. In some embodiments, the present composition contains a therapeutically effective amount of a benzodiazepine or a pharmaceutically acceptable salt thereof, and a hydroxyl group-containing compound, such as propylene glycol. In some embodiments, the present composition contains a therapeutically effective amount of a benzodiazepine or a pharmaceutically acceptable salt thereof, a Hsieh permeation enhancer, and a hydroxyl group-containing compound, such as propylene glycol.

The present composition may contain more than one type of benzodiazepine. The present compositions may be used to provide for an anxiolytic effect, an anticonvulsant effect, a sedative effect, a skeletal muscle relaxant effect, an amnesic effect or combinations thereof. The present compositions may be used alone or in combination with other compositions or methods. The present compositions may be used before, during or after other compositions or methods. For example, the present compositions may be used together with another anticonvulsant drug to treat seizure, protect against seizure, reduce or ameliorate the intensity of seizure, reduce or ameliorate the frequency of seizure, and/or prevent occurrence or re-occurrence of seizure. The present compositions may be administered by the patient or by another person (e.g., a health care professional or other person).

As used herein, “irritating” refers to the property of causing a local inflammatory response, such as reddening, swelling, itching, burning, or blistering, by immediate, prolonged, or repeated contact. For example, inflammation of a non-oral mucosa (e.g., nasal mucosa) or dermal tissue in a mammal can be an indication of irritation to that tissue. Chronic vasoconstriction, irritation, or inflammation can induce tissue damage, including ulceration, epistaxis, nasal-septal defects, and fistulae. A composition is deemed “substantially non-irritating” or “not substantially irritating,” if the composition is judged to be slightly or not irritating using any standard method for assessing dermal or mucosal irritation. Van den Donk et al., The effects of nasal drops and their additives on human nasal mucociliary clearance. Rhinology 1982; 20:127-137. Non-limiting examples of methods useful for evaluating mucosal irritation include: HET-CAM (hen's egg test-chorioallantoic membrane); slug mucosal irritation test; and in vitro tests using tissue-engineered nasal or sinus mucosa or vaginal-ectocervical tissues. Non-limiting examples of methods useful for assessing dermal irritation include the use of in vitro tests using tissue-engineered dermal tissue, such as EpiDerm® (MatTek Corp., Ashland, Mass.), which is a human skin tissue model (see, for example, Chatterjee et al., 2006 Toxicol Letters 167: 85-94) or ex vivo dermis samples. Other useful methods of irritation measurement include in vivo methods, such as dermal irritation of rat or rabbit skin (e.g., the Draize skin test (OECD, 2002, Test Guidelines 404, Acute Dermal Irritation/Corrosion) and EPA Health Effects Testing Guidelines; OPPTS 870.2500 Acute Dermal Irritation). Alternatively, human subjects receiving the present composition may be asked to score a list of various possible irritant effects from each formulation. The skilled artisan is familiar with art-recognized methods of assessing mucosal and dermal irritation. U.S. Patent Publication No. 20100198136.

Benzodiazepines have the basic structure as follows:

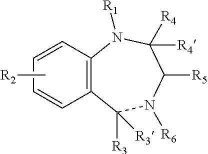

where R₁-R₆ are substituents. In some embodiments, R₁ is an optionally substituted alkyl or forms a ring with R₄, R₂ is a halogen (e.g. Cl, Br), R₃ is optionally substituted aryl (e.g. 2-Chloro or 2-Fluorophenyl), R₅ is H or OH, R₄ and R_{4′} together form a carbonyl (C═O) with the carbon to which they are attached or R₄ and R₁ form an optionally substituted heterocyclic ring with the diazepam ring atoms to which they are respectively attached; R_{3″} and R₆ together form a double bond or may be combined to form an optionally substituted heterocyclic ring along with the diazepam ring atoms to which they are respectively attached.

Non-limiting examples of benzodiazepines include diazepam, alprazolam, bentazepam, bromazepam, brotizolam, chlordiazepoxide, clobazam, clonazepam, clorazepam, clotiazepam, delorazepam, demoxazepam, estazolam, ethyl loflazepate, etizolam, fludiazepam, flumazenil, flunitrazepam, flurazepam, halazepam, ketozolam, lorazepam, loprazolam, lormetazepam, medazepam, mexazolam, midazepam, midazolam, nimetazepam, nitrazepam, nordazepam, oxazepam, pinazepam prazepam, quazepam, temazepam, tetrazepam and triazolam. The structures of some of these benzodiazepines can be found in Goodman and Gilman's The Pharmacological Basis of Therapeutics, 9th edition, McGraw Hill (1996), page 383.

In one embodiment, the benzodiazepine is diazepam (7-chloro-1-methyl-5-phenyl-1,3-dihydro-2H-1,4-benzodiazepin-2-one), which has the following structure.

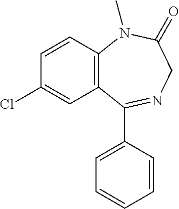 [SMILES: CN1C(=O)CN=C(c2ccccc2)c2cc(Cl)ccc21]

As used herein, the term “therapeutically effective amount” is an amount sufficient to treat a specified disorder or disease, or alternatively, to obtain a pharmacological response treating a disorder or disease. A therapeutically effective amount of benzodiazepine may vary according to factors such as the kind of benzodiazepine, desired action, physical and medical conditions of the subject, such as age, body weight, etc. A therapeutically effective amount of benzodiazepine can be determined by one of ordinary skill in the art without undue experimentation. For example, the electroencephalographic (EEG) effects of benzodiazepines may be used to determine a therapeutically effective amount of benzodiazepine. Changes in N100, P200 and P300 brain event-related potentials (ERP) elicited by auditory stimulation and electroencephalographic β-activity may be used to assess effects on neurological activity. Lindhardt et al., Electroencephalographic effects and serum concentrations after intranasal and intravenous administration of diazepam to healthy volunteers, Br. J. Clin. Pharmacol. 2001, 52, 521-527. Based on known benzodiazepine dosing regimen and the teachings herein, one skilled in the art can select the dosing regimen and dosage for a particular subject or subjects. The ability of a benzodiazepine to have a therapeutic effect can be evaluated in an animal model system predictive of efficacy in humans.

Suitable dosage formulations and methods of administering the agents can be readily determined by those of skill in the art. For example, the composition are administered at about 0.01 mg/kg to about 200 mg/kg, about 0.05 mg/kg to about 150 mg/kg, about 0.1 mg/kg to about 100 mg/kg, about 0.1 mg/kg to about 80 mg/kg, about 0.5 mg/kg to about 50 mg/kg, about 0.05 mg/kg to about 10 mg/kg, about 0.05 mg/kg to about 5 mg/kg, about 0.1 mg/kg to about 1 mg/kg, or about 0.2 mg/kg to about 0.5 mg/kg. When the compounds described herein are co-administered with another agent or therapy, the effective amount may be less than when the agent is used alone.

A therapeutically effective amount of a benzodiazepine may range from about 0.1 mg to about 50 mg, from about 1 mg to about 50 mg, from about 5 mg to about 40 mg, from about 20 mg to about 35 mg, from about 5 mg to about 30 mg, from about 0.1 mg to about 20 mg, from about 1 mg to about 20 mg, from about 0.1 mg to about 10 mg, from about 0.2 mg to about 10 mg, from about 2 mg to about 10 mg, from about 0.2 mg to about 1 mg, from about 0.5 mg to about 4 mg, about 1 to about 2 mg per dose, or from about 5 mg to about 20 mg. The benzodiazepine composition may be taken from about 1 time to about 8 times, from about 2 times to about 8 times, from about 1 time to about 3 times, or from about 4 times to about 6 times per day. One dose or more than one dose may be used to treat one episode of seizure or other disorders or conditions as mentioned above. A second dose, when required, may be given about 0.5 hour to about 72 hours, about 1 hour to about 48 hours, about 2 hours to about 48 hours, about 2 hours to about 36 hours, about 3 hours to about 24 hours, about 4 hours to about 12 hours, or about 5 hours to about 8 hours after the first dose. In certain embodiments, the present composition may be used to treat no more than 15 episodes per month, no more than 12 episodes per month, no more than 10 episodes per month, no more than 8 episodes per month, no more than 5 episodes per month, or no more than 2 episodes per month. In some embodiments, the present composition may be used to treat no more than one episode every 30 days, no more than one episode every 25 days, no more than one episode every 20 days, no more than one episode every 15 days, no more than one episode every 10 days, no more than one episode every 5 days, or no more than one episode every 2 days.

Benzodiazepine may be present in the present composition in an amount ranging from about 0.01% (w/w) to about 80% (w/w), about 0.1% (w/w) to about 50% (w/w), 0.01% (w/w) to about 15% (w/w), about 0.01% (w/w) to about 10% (w/w), about 0.1% to about 5% (w/w), about 1% (w/w) to about 30% (w/w), about 2.5% (w/w) to about 20% (w/w), from about 1% (w/w) to about 10% (w/w), from about 2% (w/w) to about 10% (w/w), from about 3% (w/w) to about 10% (w/w), or from about 4% (w/w) to about 10% (w/w).

The doses of benzodiazepine may be delivered in single or multiple doses. The doses may contain equal or different amounts of benzodiazepine.

The present compositions can be in any suitable form, including, but not limited to, solutions, suspensions, dispersions, tablets, pills, capsules, semisolids, powders, sustained release formulations, elixirs, or aerosols. The benzodiazepine compositions may contain crystalline benzodiazepine, amorphous benzodiazepine, semi-crystalline benzodiazepine, a mixture of amorphous and crystalline benzodiazepine, a mixture of amorphous and semi-crystalline benzodiazepine, a mixture of crystalline and semi-crystalline benzodiazepine a mixture of amorphous, crystalline and semi-crystalline benzodiazepine and soluble benzodiazepine.

The present composition may contain a benzodiazepine, pharmaceutically-acceptable salts thereof, or combinations thereof.

Benzodiazepine may form salts with pharmaceutically acceptable acids, such as pharmaceutically acceptable mineral acids and pharmaceutically acceptable organic acids.

Pharmaceutically acceptable mineral acids include HCl, H₂SO₄, H₂SO₃, H₃PO₄, H₃PO₃, and others that will be recognized by those of skill in the art. Pharmaceutically acceptable organic acids include acetic acid, benzoic acid, tartaric acid, citric acid, oxalic acid, maleic acid, malonic acid, etc. Non-limiting examples of the pharmaceutically acceptable acid include: 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acidascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acidfumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, benzenesulfonic acid (besylic acid), naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p) and undecylenic acid. Other pharmaceutically acceptable acids may be pharmaceutically acceptable acidic (anionic) polymers or pharmaceutically acceptable amphoteric polymers.

After administration of benzodiazepine, the plasma levels of benzodiazepine or its metabolite(s) may be assayed to determine the maximum concentration of plasma benzodiazepine (Cmax) during the selected dosing interval, the area under the plasma concentration versus time curve (AUC₀₋ₜ) and time to achieve Cmax (Tmax). AUC may be measured from time 0 to time t, where a variety of time intervals may be selected. In one embodiment, blood is collected at time 0, 5, 10, 15, 30, 45, 60 and 90 minutes after administration of nasal benzodiazepine.

A therapeutically effective plasma level of benzodiazepine may be obtained in less than about 2 hours, less than about 1.5 hours, less than about 1 hour, less than about 45 minutes, less than about 30 minutes, less than about 20 minutes, less than about 15 minutes, less than about 10 minutes, or less than about 5 minutes after administration of the composition to the patient. Tmax may be less than about 2 hours, less than about 1.5 hours, less than about 1 hour, less than about 45 minutes, less than about 30 minutes, less than about 20 minutes, less than about 15 minutes, less than about 10 minutes, or less than about 5 minutes after administration of the composition. When plasma benzodiazepine is measured from about 0 to about 2 hours after administration of a single dose of the composition, Tmax of plasma benzodiazepine may range from about 1 minute to about 10 minutes, about 2 minutes to about 8 minutes, about 5 minutes, or about 10 minutes.

The Cmax of plasma benzodiazepine after administration of the present compositions may range from about 10 to about 1000 ng/ml, from about 15 to about 800 ng/ml, from about 20 to about 500 ng/ml, from about 25 to about 400 ng/ml, from about 50 to about 800 ng/ml, from about 100 to about 600 ng/ml, from about 200 to about 500 ng/ml, from about 15 to about 300 ng/ml, from about 15 to about 200 ng/ml, from about 20 to about 180 ng/ml, from about 25 to about 125 ng/ml, from about 40 to about 110 ng/ml, from about 50 to about 110 ng/ml, from about 20 to about 50 ng/ml, or from about 40 to about 180 ng/ml.

In order to compare Cmax between compositions with different doses of benzodiazepine, Cmax may be normalized to a single dose. As used herein, the term “dose-normalized Cmax” refers to Cmax of plasma benzodiazepine dose-normalized to, for example, 1 mg. The dose-normalized Cmax of plasma benzodiazepine after administration of the present compositions may range from about 3 to about 200 ng/ml/mg, from about 5 to about 150 ng/ml/mg, from about 5 to about 100 ng/ml/mg, from about 5 to about 80 ng/ml/mg, from about 5 to about 60 ng/ml/mg, from about 3 to about 60 ng/ml/mg, from about 3 to about 10 ng/ml/mg, from about 15 to about 60 ng/ml/mg, from about 15 to about 50 ng/ml/mg, from about 18 to about 28 ng/ml/mg, or about 20 ng/ml/mg.

The AUC_{0-5 min} of plasma benzodiazepine after administration of the present compositions may range from about 10 to about 1,000 ng*min/ml, from about 20 to about 800 ng*min/ml, from about 40 to about 600 ng*min/ml, from about 50 to about 500 ng*min/ml, from about 60 to about 450 ng*min/ml, from about 60 to about 120 ng*min/ml, from about 200 to about 500 ng*min/ml, or from about 250 to about 450 ng*min/ml.

The AUC_{0-10 min} of plasma benzodiazepine after administration of the present compositions may range from about 20 to about 2,000 ng*min/ml, from about 50 to about 1,800 ng*min/ml, from about 100 to about 1,600 ng*min/ml, from about 120 to about 1,500 ng*min/ml, from about 150 to about 1,400 ng*min/ml, from about 150 to about 350 ng*min/ml, from about 150 to about 300 ng*min/ml, or from about 750 to about 1,350 ng*min/ml.

In order to compare AUC between compositions with different doses of benzodiazepine, AUC may be normalized to a single dose. As used herein, the term “dose-normalized AUC” refers to AUC of plasma benzodiazepine dose-normalized to, for example, 1 mg. The dose-normalized AUC_{0-5 min} of plasma benzodiazepine after administration of the present compositions may range from about 10 to about 500 ng*min/ml/mg, from about 20 to about 400 ng*min/ml/mg, from about 30 to about 300 ng*min/ml/mg, from about 30 to about 200 ng*min/ml/mg, from about 35 to about 150 ng*min/ml/mg, from about 40 to about 140 ng*min/ml/mg, from about 40 to about 130 ng*min/ml/mg, from about 50 to about 60 ng*min/ml/mg, or from about 40 to about 130 ng*min/ml/mg.

The dose-normalized AUC_{0-10 min} of plasma benzodiazepine after administration of the present compositions may range from about 20 to about 1000 ng*min/ml/mg, from about 40 to about 800 ng*min/ml/mg, from about 60 to about 600 ng*min/ml/mg, from about 60 to about 400 ng*min/ml/mg, from about 70 to about 300 ng*min/ml/mg, from about 80 to about 2800 ng*min/ml/mg, from about 100 to about 1200 ng*min/ml/mg, or from about 80 to about 2600 ng*min/ml/m, from about 10 to about 500 ng*min/ml/mg, from about 20 to about 400 ng*min/ml/mg, from about 30 to about 300 ng*min/ml/mg, from about 30 to about 200 ng*min/ml/mg, from about 35 to about 150 ng*min/ml/mg, from about 40 to about 140 ng*min/ml/mg, from about 40 to about 130 ng*min/ml/mg, from about 50 to about 60 ng*min/ml/mg, or from about 40 to about 130 ng*min/ml/mg.

The plasma benzodiazepine may be measured during a period ranging from about 0 to about 48 hours, from about 0 to about 24 hours, from about 0 to about 12 hours, from about 0 to about 120 minutes, from about 0 minutes to about 90 minutes, from about 0 minutes to about 60 minutes, or from about 0 minutes to about 30 minutes after administration of the present composition.

The bioavailability (F %) of the present composition may be calculated using the following equation:

$$F ( \% ) = \frac{{AUC}_{IN}}{{AUC}_{IV}} \times \frac{{Dose}_{IV}}{{Dose}_{IN}} \times 100$$

where “IN” stands for intranasal, “IV” intravenous.

Similarly, relative bioavailability (F_{R}%) of the present composition compared with a reference formulation (“RF”) may be calculated as follows:

$${F}_{R} ( \% ) = \frac{{AUC}_{IN}}{{AUC}_{RF}} \times \frac{{DOSE}_{RF}}{{DOSE}_{IN}} \times 100$$

For example, relative bioavailability (F_{R}%) of the present composition compared with a rectal formulation (“RC”) may be calculated as below:

$${F}_{R} ( \% ) = \frac{{AUC}_{IN}}{{AUC}_{RC}} \times \frac{{DOSE}_{RC}}{{DOSE}_{IN}} \times 100$$

Relative bioavailability of the present composition compared with a rectal formulation may range from about 50% to about 500%, from about 60% to about 450%, from about 70% to about 400%, from about 80% to about 350%, from about 90% to about 300%, from about 90% to about 150%, from about 100% to about 500%, from about 120% to about 500%, from about 150% to about 400%, from about 200% to about 300%, from about 250% to about 300%, from about 100% to about 200%, from about 100% to about 150%, or from about 100% to about 120%.

The blood (or plasma) concentration of benzodiazepine may be assayed by gas-liquid chromatography, optionally coupled with electron-capture detection (GLC/EC) [J. A. Zingales: Diazepam metabolism during chronic medication, unbound fraction in plasma, erythrocytes and urine. J. Chromatogr. 75:55-78 (1975); D. M. Hailey: Chromatography of the 1,4-benzodiazepines. J. Chromatogr. 98: 527-568, 1974; and M. Linnoila and F. Dorrity: Rapid gas chromatographic assay of serum diazepam, N-desmethyldiazepam, and N-desalkylfurazepam. Acta pharmacol. et. toxicol. 41: 458-464, 1977]. An enzyme-immunoassay (EIA) based analysis for benzodiazepine in serum may be used (see, for example, an EIA assay for diazepam from STC Technologies, Inc., Bethlehem, Pa., USA). Lindhardt et al., Intranasal bioavailability of diazepam in sheep correlated to rabbit and man. International Journal of Pharmaceutics, 231 (2002) 67-72. Lindhardt et al., 2001, Electroencephalographic effects and blood levels after intranasal administration of diazepam to humans. Br. J. Clin. Pharm. 52, 1-12. Radioreceptor assays may also be used. U.S. Pat. No. 4,239,744. Non-limiting examples of other methods to measure blood (or plasma) concentration of benzodiazepine include, but are not limited to, gas chromatography (GC), liquid chromatography (LC), high pressure liquid chromatography (HPLC), nuclear magnetic resonance (NMR) spectroscopy, mass spectrometry (MS), LC/MS, LC/MS/MS, HPLC-MS, GC-MS, infrared spectroscopy (IR), and thin layer chromatography (TLC).

The present composition may be administered by any method known in the art, including, without limitation, intranasal, pulmonary, oral, transdermal, ocular, intraperitoneal, inhalation, intravenous, ICV, intracisternal injection or infusion, subcutaneous, implant, vaginal, sublingual, urethral (e.g., urethral suppository), subcutaneous, intramuscular, intravenous, rectal, sub-lingual, mucosal, ophthalmic, spinal, intrathecal, intra-articular, intra-arterial, sub-arachinoid, bronchial and lymphatic administration. Topical formulation may be in the form of gel, ointment, cream, aerosol, etc; intranasal formulation can be delivered as a spray or in a drop; transdermal formulation may be administered via a transdermal patch or iontorphoresis; inhalation formulation can be delivered using a nebulizer or similar device. Compositions can also take the form of tablets, pills, capsules, semisolids, powders, sustained release formulations, solutions, suspensions, elixirs, aerosols, or any other appropriate compositions.

In some embodiments, a composition of the present invention is administered to a subject via one or more than one route. For example, a subject may benefit from receiving mucosal administration (e.g., nasal administration) and, additionally, receiving one or more other routes of administration (e.g., parenteral or pulmonary administration).

To prepare the present pharmaceutical compositions, one or more benzodiazepine may be mixed with a pharmaceutical acceptable carrier, adjuvant and/or excipient, according to conventional pharmaceutical compounding techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers and adjuvants, see Remington's Pharmaceutical Sciences, edited by E. W. Martin (Mack Publishing Company, 18th ed., 1990). The compositions also can include stabilizers and preservatives.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer. If the composition is in the form of a gel, the composition may be rubbed onto a membrane of the patient, for example, the skin, preferably intact, clean, and dry skin, of the shoulder or upper arm and or the upper torso, and maintained thereon for a period of time sufficient for delivery of the benzodiazepine to the blood serum of the patient. The composition of the present invention in gel form may be contained in a tube, a sachet, or a metered pump. Such a tube or sachet may contain one unit dose, or more than one unit dose, of the composition. A metered pump may be capable of dispensing one metered dose of the composition.

This invention also provides the compositions as described above for ocular administration. For ocular administration, the compositions described herein can be formulated as a solution, emulsion, suspension, etc. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

Compositions according to the invention may be administered to mucosal membranes for example in the nose, vagina, rectum, ears, eyes, oral cavity, lungs, genito-urinary tracts, and gastro-intestinal tract.

In certain embodiments, the present compositions may be for intranasal administration. The benzodiazepine may be administered intranasally in a liquid form such as a solution, an emulsion, a suspension, drops, or in a solid form such as a powder, gel, or ointment. Wermeling, D. P., Intranasal Delivery of Antiepileptic Medications for Treatment of Seizures, Neurotherapeutics, Vol. 6, No. 2, 2009. In one embodiment, the present composition is a microemulsion system comprising ethyl laurate, Tween 80, propylene glycol, ethanol and H₂O. Li et al., Development of an ethyl laurate-based microemulsion for rapid-onset intranasal delivery of diazepam, International Journal of Pharmaceutics, 237 (2002) 77-85. In another embodiment, the present composition is a microemulsion comprising ethyl laurate, Caprylcaproyl macrogol-8 glyceride (Labrasol®), diethylene glycol monoethyl ether (Transcutol®P), ethanol, and H₂O. Kaur et al., Pharmacokinetics and brain uptake of diazepam after intravenous and intranasal administration in rats and rabbits, International Journal of Pharmaceutics, 364 (2008) 27-35.

The pharmaceutical composition can be administered intranasally by devices including, but not limited to, an intranasal spray device, an atomizer, a nebulizer, a metered dose inhaler (MDI), a dry powder inhaler (DPI), a pressurized dose inhaler, an insufflator, an intranasal inhaler, a nasal spray bottle, a unit dose container, a pump, a dropper, a squeeze bottle, an aerosolized metered dose pumps, a manual metered dose pump, a metered dose spray-producing squeeze bottles, a multi-dose bottle, or a bi-directional device. Nasal sprays may be liquid or solid nasal sprays. The present composition may be administered as aerosols or in non-aerosol forms. The nasal delivery device can be metered to administer an accurate effective dosage amount to the nasal cavity. The nasal delivery device can be for single unit delivery or multiple unit delivery. The compounds of the present invention may also be delivered through a tube, a catheter, a syringe, a packtail, a pledget, a nasal tampon or by submucosal infusion. U.S. Patent Publication Nos. 20090326275, 20090291894, 20090281522 and 20090317377. In some embodiments, e.g., in the case of an unconscious patient experiencing a seizure, the aerosolized metered dose pump connected to a close fitting plastic mask covering the nose and mouth can be used.

The benzodiazepine may be formulated with or without solvents, and formulated with or without carriers. The formulation may be a solution, or may be an emulsion with one or more surfactants.

The benzodiazepine can be formulated as aerosols using standard procedures. For example, an aerosol spray may be generated from pressurized container with a suitable propellant such as, dichlorodifluoromethane, trichlorofluoromethane, dichlorotetrafluoroethane, hydrocarbons, compressed air, nitrogen, carbon dioxide, or other suitable gas. The dosage unit can be determined by providing a valve to deliver a metered amount. Pump spray dispensers can dispense a metered dose or a dose having a specific particle or droplet size. As used herein, the term “aerosol” may refer to a suspension or dispersion of either liquid droplets or solid powder in air (or in a gas). Liquid droplets may be formed from solutions, suspensions and dispersions of drug in a liquid, such as water or a non-aqueous solvent. The aerosol may be insufflated or inhaled through the nose. Aerosols may be produced in any suitable device, such as an MDI, a nebulizer, or a mist sprayer. Gonda (1990) Critical Reviews in Therapeutic Drug Carrier Systems 6:273-313. Raeburn et al., (1992) Pharmacol. Toxicol. Methods 27:143-159.

An aerosol according to the invention may be insufflated using a suitable mechanical apparatus. In some embodiments, the apparatus may include a reservoir and sprayer, which is a device adapted to expel the pharmaceutical dose in the form of a spray. A number of doses of the drug to be administered may be contained within the reservoir, optionally in a liquid solution or suspension or in a solid particulate formulation, such as a solid particulate mixture.

Nebulizer devices produce a stream of high velocity air that causes a therapeutic agent in the form of liquid to spray as a mist. The therapeutic agent is formulated in a liquid form such as a solution or a suspension of particles of suitable size. In one embodiment, the particles are micronized. The term “micronized” is defined as having about 90% or more of the particles with a diameter of less than about 10 μm. Suitable nebulizer devices are provided commercially, for example, by PARI GmbH (Starnberg, Germany). Other nebulizer devices include Respimat (Boehringer Ingelheim) and those disclosed in, for example, U.S. Pat. Nos. 7,568,480 and 6,123,068, and WO 97/12687. The benzodiazepine can be formulated for use in a nebulizer device as an aqueous solution or as a liquid suspension.

DPI devices typically administer a therapeutic agent in the form of a free flowing powder that can be dispersed in a patient's air-stream during inspiration. DPI devices which use an external energy source may also be used in the present invention. In order to achieve a free flowing powder, the therapeutic agent can be formulated with a suitable excipient (e.g., lactose). A dry powder formulation can be made, for example, by combining dry lactose having a particle size between about 1 μm and 100 μm with micronized particles of the benzodiazepine and dry blending. Alternatively, the benzodiazepine can be formulated without excipients. The formulation is loaded into a dry powder dispenser, or into inhalation cartridges or capsules for use with a dry powder delivery device. Examples of DPI devices provided commercially include Diskhaler (GlaxoSmithKline, Research Triangle Park, N.C.) (see, e.g., U.S. Pat. No. 5,035,237); Diskus (GlaxoSmithKline) (see, e.g., U.S. Pat. No. 6,378,519; Turbuhaler (AstraZeneca, Wilmington, Del.) (see, e.g., U.S. Pat. No. 4,524,769); and Rotahaler (GlaxoSmithKline) (see, e.g., U.S. Pat. No. 4,353,365). Further examples of suitable DPI devices are described in U.S. Pat. Nos. 5,415,162, 5,239,993, and 5,715,810 and references therein.

MDI devices typically discharge a measured amount of therapeutic agent using compressed propellant gas. Formulations for MDI administration include a solution or suspension of active ingredient in a liquefied propellant. Examples of propellants include hydrofluoroalklanes (HFA), such as 1,1,1,2-tetrafluoroethane (HFA 134a) and 1,1,1,2,3,3,3-heptafluoro-n-propane, (HFA 227), and chlorofluorocarbons, such as CCl₃F. Additional components of HFA formulations for MDI administration include co-solvents, such as ethanol, pentane, water; and surfactants, such as sorbitan trioleate, oleic acid, lecithin, and glycerin. (See, for example, U.S. Pat. No. 5,225,183, EP 0717987, and WO 92/22286). The formulation is loaded into an aerosol canister, which forms a portion of an MDI device. Examples of MDI devices developed specifically for use with HFA propellants are provided in U.S. Pat. Nos. 6,006,745 and 6,143,227. For examples of processes of preparing suitable formulations and devices suitable for inhalation dosing see U.S. Pat. Nos. 6,268,533, 5,983,956, 5,874,063, and 6,221,398, and WO 99/53901, WO 00/61108, WO 99/55319 and WO 00/30614.

The benzodiazepine may be encapsulated in liposomes or microcapsules for delivery via inhalation. A liposome is a vesicle composed of a lipid bilayer membrane and an aqueous interior. The lipid membrane may be made of phospholipids, examples of which include phosphatidylcholine such as lecithin and lysolecithin; acidic phospholipids such as phosphatidylserine and phosphatidylglycerol; and sphingophospholipids such as phosphatidylethanolamine and sphingomyelin. Alternatively, cholesterol may be added. A microcapsule is a particle coated with a coating material. For example, the coating material may consist of a mixture of a film-forming polymer, a hydrophobic plasticizer, a surface activating agent or/and a lubricant nitrogen-containing polymer. U.S. Pat. Nos. 6,313,176 and 7,563,768.

The present composition may be delivered by a pump sprayer that includes a metering pump. In some embodiments, the apparatus includes a pressurized spray device, in which the sprayer includes a metering valve and the pharmaceutical composition further comprises a pharmaceutically acceptable propellant. Exemplary propellants include one or mixture of chlorofluorocarbons, such as dichlorodifluoromethane, as well as hydrofluorocarbons, such as 1,1,1,2-tetrafluoroethane (HFC-134a) and 1,1,1,2,3,3,3-heptafluoropropane (HFC-227). Pharmaceutically acceptable propellants also include, but are not limited to, ethyl chloride, butane, propane, dichlorotetrafluoroethane, and trichloromonofluoromethane.

Powders can be administered using a nasal insufflator. The benzodiazepine may be delivered to the nasal cavity as a powder in a form such as microspheres. The benzodiazepine may be absorbed to a solid surface, for example, a carrier. The powder or microspheres may be administered in a dry, air-dispensable form. The powder or microspheres may be stored in a container of the insufflator. Alternatively the powder or microspheres may be filled into a capsule, such as a gelatin capsule, or other single dose unit adapted for nasal administration.

In one embodiment, the composition of the present invention is delivered through a nasal spray applicator. The composition may be placed in an intranasal spray-dosing device or atomizer and may be applied by spraying it into the nostrils of a subject for delivery to the mucous membrane of the nostrils. A sufficient amount is applied to achieve the desired systemic or localized benzodiazepine levels. For an intranasal spray, an application of up to about 200 microliters, from about 50 to about 150 microliters, or from about 75 to about 120 microliters may be applied. One or more nostrils may be dosed and application may occur as often as desired or as often as is necessary.

In one embodiment, a first dose of benzodiazepine in 100 microliters is administered into the nostril. After an appropriate time period when the amount of liquid has been absorbed, a second dose may be administered sequentially into the same nostril, or may be administered into a different nostril. More dose(s) may be administered thereafter. In one embodiment, the second dose may be administered within 1-5 seconds after administration of the first dose. The amount of benzodiazepine in the first or subsequent dose(s) will be determined clinically and may vary.

In some embodiments, the administration of the composition comprises spraying at least a portion of the therapeutically effective amount of the composition into at least one nostril. In some embodiments, the administration of the composition comprises spraying at least a portion of the therapeutically effective amount of the composition into each nostril. In some embodiments, the administration of the composition comprises spraying a first quantity of the composition into the first nostril, spraying a second quantity of the composition into a second nostril, and optionally after a pre-selected time delay, spraying a third quantity of the composition into the first nostril. Some embodiments further comprise, optionally after a pre-selected time delay, administering at least a fourth quantity of the composition to the second nostril.

In some embodiments, additional metered sprays are applied to alternating nostrils until the full target therapeutic dose has been administered to the patient. In some embodiments, there is a time increment of from several seconds to 5 minutes, preferably about 10 seconds to about 1 minute, between applications of benzodiazepine drug to the same nostril. This allows time for the drug to cross the nasal mucosa and enter the blood stream. Multiple applications of metered sprays to each nostril, optionally separated by a time interval, allows administration of a full therapeutic dose in increments small enough to permit full absorption of the benzodiazepine drug into the blood stream and avoid loss of drug down the back of the throat.

The pharmaceutical composition can be delivered to the nasal cavity by direct placement of the composition in the nasal cavity, for example, in the form of a gel, an ointment, a nasal emulsion, a lotion, a cream, a nasal tampon, a dropper, or a bioadhesive strip. In certain embodiments, it can be desirable to prolong the residence time of the pharmaceutical composition in the nasal cavity, for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin, or derivatives thereof), gelatin, a liposome, carbamer, polyvinyl alcohol, alginate, acacia, chitosans and/or cellulose (e.g., methyl or propyl; hydroxyl or carboxy; carboxymethyl or hydroxylpropyl).

The composition containing the benzodiazepine can be administered by oral inhalation into the respiratory tract, i.e., the lungs.

Pulmonary drug delivery requires aerosolization of a solid or liquid and delivery of the aerosol to the lungs via the mouth and throat. Particles of medicament may be administered to the lungs as dry powder aerosols or liquid aerosols. Dry powder aerosols are generally administered to the lungs with dry powder inhaler (DPI) inhalation devices. Dry powder inhalers can include breath actuated dry powder inhalers, such as are described in U.S. Pat. No. 7,434,579. Metered-dose inhalers contain medicament suspended in a propellant, a mixture of propellants, or a mixture of solvents, propellants, and/or other excipients in compact pressurized aerosol dispensers. An MDI product may discharge up to several hundred metered doses of medicament. Each actuation may contain from a few micrograms (mcg) up to milligrams (mg) of the active ingredients delivered in a volume typically between 25 and 100 microliters.

Another type of liquid aerosol dispersion device is nebulizer, which uses a jet, a vibrating mesh or other means to aerolsolize a suspension containing particles of medicament.

The benzodiazepine composition can be given alone or in combination with other drugs for the treatment of a disorders or condition for a short or prolonged period of time. For example, the present composition may be administered together with the following non-limiting exemplary drugs: paraldehyde; aromatic allylic alcohols (such as stiripentol); barbiturates (e.g. phenobarbitol, primidone, methylphenobarbital, metharbital and barbexaclone); bromides (such as potassium bromide); carbamates (such as felbamate); carboxamides (such as carbamazepine and oxcarbazepine); fatty acids (such as valproic acid, sodium valproate, and divalproex sodium, vigabatrin, progabide, tiagabine); fructose, topiramate, GABA analogs (e.g. gabapentin and pregabalin); hydantoins (e.g. ethotoin, phenyloin, mephenyloin and fosphenyloin); oxazolidinediones (such as paramethadione, trimethadione, ethadione); propionates (e.g. beclamide), pyrimidinediones (e.g. primidone); pyrrolidines (e.g. brivaracetam, levetiracetam and seletracetam); succinimides (e.g. ethosuximide, phensuximide and mesuximide); sulfonamides (e.g. acetazolamide, sulthiame, methazolamide and zonisamide); triazines (such as lamotrigine); ureas (such as pheneturide, phenacemide); valproylamides (such as valpromide and valnoctamide); or pharmaceutically acceptable salts or combinations thereof.

A benzodiazepine may also be co-administered with antiviral agents, anti-inflammatory agents or antibiotics. The agents may be administered concurrently or sequentially. A benzodiazepine can be administered before, during or after the administration of the other active agent(s).

The present compositions can be administered to a mammal, preferably a human. Mammals include, but are not limited to, a rodent (e.g., a guinea pig, a hamster, a rat, a mouse), a primate, a marsupial (e.g., kangaroo, wombat), a monotreme (e.g., duckbilled platypus), murine (e.g., a mouse), a lagomorph (e.g., a rabbit), canine (e.g., a dog), feline (e.g., a cat), equine (e.g., a horse), porcine (e.g., a pig), ovine (e.g., a sheep), bovine (e.g., a cow), simian (e.g., a monkey or ape), a monkey (e.g., marmoset, baboon), an ape (e.g., gorilla, chimpanzee, orangutang, gibbon).

The present composition may contain a benzodiazepine and a permeation enhancer. For example, for intranasal delivery, the permeation enhancer can enhance the permeation of benzodiazepine through the nasal mucosa.

In some embodiments, compounds containing one or more than one hydroxyl group may be used as permeation enhancers. Some of these hydroxyl group-containing compounds can also serve as solvents in the composition.

Non-limiting examples of hydroxyl group-containing compounds that may be used as permeation enhancers include alcohols (such as ethanol), diols (such as propylene glycol also known as 1,2-propanediol; 1,3-propanediol; butylene glycol including 1,3-butanediol, 1,2-butanediol, 2,3-butanediol, and 1, 4 butanediol; hexylene glycol; dipropylene glycol, 1,5-pentanediol, 1,2-pentanediol, 1,8-octanediol, etohexadiol, p-menthane-3,8 diol, 2-methyl-2,4-pentanediol), triols (such as glycerin), polyols (such as suitable polymers containing multiple hydroxyl groups, including polyethylene glycols or PEGs, polypropylene glycols, polysorbates, and sorbitan esters; and suitable sugar alcohols), cyclitols (such as pinitol, insoitol), cyclic diols (such as cyclohexane diol), aromatic diols (such as hydroquinone, bisphenol A, resorcinol and catechol).

In certain embodiments, the general formula of the permeation enhancer that may be used in the present compositions is shown below.

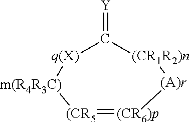

wherein X and Y are oxygen, sulfur or an imino group of the structure

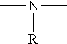

or ═N—R with the proviso that when Y is the imino group, X is an imino group, and when Y is sulfur, X is sulfur or an imino group, A is a group having the structure

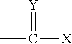

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11, and said compound will enhance the rate of the passage of an antigen (or an antibody) across body membranes. Hereinafter these compounds are referred to as enhancers. When R, R₁, R₂, R₃, R₄, R₅ or R₆ is alkyl it may be methyl, ethyl, propyl, isopropyl, butyl, isobutyl, sec-butyl, amyl, hexyl, and the like. Such permeation enhancers are described in U.S. Pat. Nos. 5,023,252 and 5,731,303.

Preferably, the permeation enhancer compounds of this invention are the cyclic lactones (the compounds wherein both X and Y are oxygen, (q is 1 and r is 0), the cyclic diesters (the compounds wherein both X and Y are oxygen, and both q and r are 1), and the cyclic ketones (the compounds wherein both q and r are 0 and Y is oxygen). In the cyclic diesters m+n is preferably at least 3. In the cyclic ketones m+n is preferably from 11 to 15 and p is preferably 0.

Enhancers of the above structural formula are referred to herein as “Hsieh enhancers” and are described, for example, in aforementioned U.S. Pat. Nos. 5,023,252 and 5,731,303 (hereinafter the “Hsieh Patents”). Such enhancers are lipophilic and are “membrane-compatible,” meaning that they do not cause damage to the membrane on which the composition of the present invention is to be applied (hereinafter the “target membrane”). Such enhancers also produce a low level of irritability or no irritability to the target membrane, and in fact may serve as emollients.

Preferred enhancers for use in the present invention are macrocyclic enhancers. The term “macrocyclic” is used herein to refer to cyclic compounds having at least 12 carbons in the ring. Examples of preferred macrocyclic enhancers for use in the present invention include: (A) macrocyclic ketones, for example, 3-methylcyclopentadecanone (muscone), 9-cycloheptadecen-1-one (civetone), cyclohexadecanone, and cyclopentadecanone (normuscone); and (B) macrocyclic esters, for example, pentadecalactones such as oxacyclohexadecan-2-one (cyclopentadecanolide, ω-pentadecalactone).

One of ordinary skill in the art would recognize that the instant teachings would also be applicable to other permeation enhancers. Non-limiting examples of other permeation enhancers useful in the instant invention are the simple long chain esters that are Generally Recognized As Safe (GRAS) in the various pharmacopoeial compendia. These may include simple aliphatic, unsaturated or saturated esters. Non-limiting examples of such esters include isopropyl myristate, isopropyl palmitate, myristyl myristate, octyl palmitate, and the like. Non-limiting examples other permeation enhancers include alcohols (e.g., short- and long-chain alcohols), polyalcohols, amines and amides, urea, amino acids and their esters, amides, azone or pyrrolidone and its derivatives, terpenes, fatty acids and their esters, macrocyclic compounds, sulfoxides, tensides, benzyldimethylammonium chloride, cetyl trimethyl ammonium bromide, cineole, cocamidopropyl betaine, cocamidopropyl hydroxysultaine, dodecyl pyridinium chloride, dodecylamine, hexadecyl trimethylammoniopropane sulfonate, limonene, linoleic acid (OA), linolenic acid (LA), menthol, methyl laurate, methylpyrolidone, N-decyl-2-pyrrolidone, NLS, nicotine sulfate, nonyl-1,3-dioxolane, octyl trimethylammonium bromide, oleyl betaine, PP, polyethyleneglycol dodecyl ether, polyoxyethelene sorbitan monolaurate (Tween 20, or Polysorbate 20), SLA, sodium oleate, sodium lauryl sulfate, sodium octyl sulfate (SOS), sorbitan monolaurate (S20), tetracaine, and Triton X-100. Additional examples of permeation enhancers can be found in Sayani et al., Crit. Rev. Ther. Drug Carrier Syst. 13:85-184 (1996); Karande et al: Nature Biotechnology 22, (2), 192-197, (2004); Pfister et al., Med Device Technol. November-December 1990, 1(6):28-33; Mitragotri, Pharm Res. November 2000; 17(11):1354-9; and Hadgraf, Int J. Pharm. 1999; 184(1):1-6, and references cited therein. U.S. Pat. No. 7,883,718. The enhancers should be suitable for use in a pharmaceutical composition. The artisan of ordinary skill will also appreciate that those materials that are incompatible with or irritating to mucous membranes should be avoided.

The enhancer may be present in the composition in a concentration effective to enhance penetration of the pharmaceutically active agent that is to be delivered through the nasal mucosa. Various considerations should be taken into account in determining the amount of enhancer to use. Such considerations include, for example, the amount of flux (rate of passage through the membrane) achieved and the stability and compatibility of the components in the formulations. The enhancer may be used in an amount of greater than about 20%, greater than about 30%, greater than about 40%, greater than about 50%, greater than about 60%, greater than about 70%, greater than about 80%, greater than about 90%, about 0.001% (w/w) to about 99.9% (w/w), about 0.01% (w/w) to about 90% (w/w), about 0.1 to about 80% (w/w), about 1% to about 70% (w/w), about 5% (w/w) to 60% (w/w), about 1.0% (w/w) to about 3% (w/w), about 10% (w/w) to about 20% (w/w), about 60% (w/w) to about 98% (w/w), about 65% (w/w) to about 98% (w/w), or about 80% (w/w) to about 98% (w/w), about 60% (w/w) to about 95% (w/w), about 65% (w/w) to about 90% (w/w), or about 80% (w/w) to about 95% (w/w) of the composition.

In forming an emulsion in which the water-insoluble enhancer is a normally solid material, the enhancer is dissolved in a suitable solvent. If the enhancer is a normally liquid material which is water-immiscible, a suitable solvent for the enhancer may or may not be used. In certain embodiments, the enhancer is dissolved, dispersed, suspended, or solubilized in suitable solvent(s) such as alcohols, oils, glycerol, ethylene glycol, propylene glycol, hexane, acetone, freon, water, other polar or non-polar solvents, or a mixture, which is then added to a composition comprising an effective amount of the desired benzodiazepine. In some cases, when the enhancers are in the liquid form, a “neat” solution of enhancer can be directly incorporated in the benzodiazepine, pharmaceutical carrier, and enhancer mixture, in which the concentration of enhancer ranges from about 0.1% to about 99.9% (w/w).

In some embodiments, the present composition may contain one or more than one permeation enhancer. For example, the composition can comprise a Hsieh permeation enhancer, and a hydroxyl group-containing compound.

Examples of pharmaceutically-acceptable solvents that may be used in the present composition may be found in reference books such as the Handbook of Pharmaceutical Excipients (Fifth Edition, Pharmaceutical Press, London and American Pharmacists Association, Washington, 2006). Non-limiting examples of pharmaceutically-acceptable solvents that may be used in the present composition include, but are not limited to, propylene glycol (also known as 1,2-dihydroxypropane, 2-hydroxypropanol, methyl ethylene glycol, methyl glycol or propane-1,2-diol), ethanol, methanol, propanol, isopropanol, butanol, glycerol, polyethylene glycol (PEG), glycol, Cremophor EL or any forms of polyethoxylated castor oil, dipropylene glycol, dimethyl isosorbide, propylene carbonate, N-methylpyrrolidone, glycofurol, tetraethyleneglycol, propylene glycol fatty acid esters, and mixtures thereof. Ivaturi et al., Bioavailability and tolerability of intranasal diazepam in healthy adult volunteers. Epilepsy Res. 2009, 84(2-3):120-6. Hou et al., Enhanced Permeation of Diazepam through Artificial Membranes from Supersaturated Solutions, J. of Pharmaceutical Sciences, Vol. 95, NO. 4, 2006. In one embodiment, the present composition comprises a therapeutically effective amount of benzodiazepine and propylene glycol. Benzodiazepine may be formulated in a non-aqueous delivery vehicle, such as hydrofluorocarbon propellants, hydrocarbon propellants, etc.

In one embodiment, the present composition contains a therapeutically effective amount of a benzodiazepine or a pharmaceutically acceptable salt thereof, and propylene glycol.

The amount of a solvent may be greater than about 10% (w/w), greater than about 20% (w/w), greater than about 30% (w/w), greater than about 40% (w/w), greater than about 50% (w/w), greater than about 60% (w/w), greater than about 70% (w/w), greater than about 80%, (w/w), or greater than about 90% (w/w). In certain embodiments, propylene glycol is greater than about 30% (w/w), greater than about 50% (w/w), or greater than about 80% (w/w).

Pharmaceutical carriers include suitable non-toxic vehicles in which benzodiazepine is dissolved, dispersed, impregnated, or suspended, such as water or other solvents, fatty materials, celluloses and their derivatives, proteins and their derivatives, collagens, gelatine, polymers, adhesives, sponges, fabrics, and the like. Pharmaceutical carriers also include excipients which are added to provide better solubility or dispersion of the drug in the vehicle. Such excipients may include non-toxic surfactants, solubilizers, emulsifiers, chelating agents, binding materials, lubricants softening agents, and the like.

A liquid carrier may be present in the composition in a concentration effective to serve as a suitable vehicle for the compositions of the present invention. In general, the carrier is used in an amount of about 40 to about 98 wt. %, or about 50 to about 98 wt. % of the composition. The compositions of the present invention are preferably delivered as nasal sprays.

The liquid carrier may be water or any other suitable liquid, solvent, or mixture thereof. Benzodiazepine may be dispersed or dissolved in the liquid carrier in a therapeutically effective amount. The water may contain suitable buffering agents to result in a pH wherein the particular benzodiazepine is delivered optimally, or it may contain other carriers, such as glycerin, propylene glycol, polyethylene glycols of various sizes, amino acid modifiers, such as arginine and the like, and other suitable soluble excipients, as is known to those who are proficient in the art of compounding or pharmaceutics.

In certain embodiments, the present composition takes the form of an emulsion, for example, an oil-in-water emulsion, a water-in-oil emulsion, and a water-in-oil-in-water emulsion. The active compounds of the compositions of the present invention may exist in either the continuous or the dispersed phase or in both phases depending upon whether the compounds are hydrophilic, lipophilic, or amphiphilic. The benzodiazepine emulsions may be achieved in known manner, for example, by mixing the benzodiazepine-containing “oil phase” with an appropriate aqueous phase, e.g., water, saline or buffer solutions. Methods and appropriate aqueous media for obtaining emulsions are well known in the art.

The composition of the present invention may also comprise an emulsifying agent for use in aiding the formation of an emulsion. Essentially any suitable hydrocolloid emulsifying agent, typically a solid material, or a mixture of two or more such emulsifying agents can be used in the practice of the present invention. Hydrocolloid emulsifying agents include: vegetable derivatives, for example, acacia, tragacanth, agar, pectin, and carrageenan; animal derivatives, for example, gelatin, lanolin, cholesterol, and lecithin; semi-synthetic agents, for example, methylcellulose and carboxymethylcellulose; and synthetic agents, for example, acrylic emulsifying agents such as carbomers. The hydrocolloid emulsifying agent forms hydrocolloids (hydrated lyophilic colloids) around the emulsified liquid droplets of the emulsion. The hydrocolloid serves as a protective layer around each emulsified droplet which physically repulses other droplets, thus hindering Ostwald ripening (the tendency of emulsified droplets to aggregate). In contrast, other emulsifying agents typically protect the emulsified droplets by forming a liquid crystalline layer around the emulsified droplets. In compositions which employ a liquid crystalline layer-forming emulsifying agent, the hydrophilic-lipophilic balance (HLB) of the oil phase of the emulsion must be matched with that of the emulsifying agent to form a stable emulsion and, often, one or more additional emulsifying agents (secondary emulsifying agents) must be added to further stabilize the emulsion. The aforementioned liquid crystalline layer also retards the release of the compounds of the dispersed phase upon contact with the target substrate.

The hydrocolloid emulsifying agents for use in the composition of the present invention include compounds which exhibit a low level of irritability or no irritability to the target membrane and which have good bioadhesive and mucoadhesive properties. Examples of hydrocolloid emulsifying agents which exhibit such properties include cellulosic emulsifying agents and acrylic emulsifying agents, including, for example, those which have an alkyl group containing from about 10 to about 50 carbon atoms. Particularly preferred acrylic emulsifying agents for use in the present invention are copolymers of a carboxylic acid and an acrylic ester (described, for example, in U.S. Pat. No. 3,915,921 to Schlatzer and U.S. Pat. No. 4,509,949 to Huang et al.), with those which are cross-linked being especially preferred. An example of such an especially preferred emulsifying agent for use in forming an oil-in-water emulsion is “acrylates/C₁₀₋₃₀ alkyl acrylate crosspolymer”, a cross-linked polymer of acrylic acid and (C₁₀₋₃₀) alkyl acrylates. Acrylates/C₁₀₋₃₀ alkyl acrylate crosspolymer is available from Noveon, Inc. (previously B.F. Goodrich) and is sold under the trade name Pemulen®. Acrylates/C₁₀₋₃₀ alkyl acrylate crosspolymer has a small lipophilic portion and a large hydrophilic portion, thus allowing for it to function as a primary emulsifier for the formation of oil-in-water emulsions. In addition, acrylates/C₁₀₋₃₀ alkyl acrylate crosspolymer is capable of releasing the compounds of the dispersed phase upon contact with a substrate, namely, biological membranes or mucosa and will not re-wet (the oil phase will not re-emulsify upon contact with water). Additional information regarding acrylates/C₁₀₋₃₀ alkyl acrylate crosspolymer, which is listed in the U.S. Pharmacopeia, is provided in Noveon publications TDS-114, 117, 118, 124, 232-3, and 237, and PDS Pemulen 1622.

In forming an emulsion in which the water-insoluble enhancer is a normally solid material, the enhancer is dissolved in a suitable solvent. If the enhancer is a normally liquid material which is water-immiscible, a suitable solvent for the enhancer may or may not be used, as appropriate.

The emulsifying agent is present in the composition in a concentration that is effective to form the desired liquid emulsion. In general the emulsifying agent is used in an amount of about 0.001 to about 5 wt. % of the composition, and more generally in an amount of about 0.01 to about 5 wt. % of the composition, and most generally in an amount of about 0.1 to about 2 wt. % of the composition.

The composition of the present invention may include, as an optional ingredient, particulate solids dispersed in the composition. For example, the composition may include an additional pharmaceutically-active compound dispersed in the liquid continuous phase of the emulsion in the form of microcrystalline solids or nanoparticulates.

While the hydrocolloid emulsifying agent forms a protective layer around the emulsified liquid droplets, thus forming a stable emulsion by hindering Ostwald-ripening without the need for further stabilizing agents, in some instances it may be desirable to further improve the stability of the emulsion. Such may be accomplished by the addition of Ostwald-ripening inhibitors and/or surfactants.

An Ostwald-ripening inhibitor is a material which reduces the tendency of emulsified droplets to aggregate and form larger droplets. Essentially any suitable Ostwald-ripening inhibitor or a mixture of such inhibitors may be used to improve further the physical stability of the emulsion. Preferred Ostwald-ripening inhibitors are hydrophobic agents such as hydrocarbons and hydrocarbon waxes. Examples of hydrophobic agents are petrolatum, hexadecane, and long-chain esters, for example, octyl palmitate. The Ostwald-ripening inhibitor is present in the composition in a concentration effective to prevent the emulsified droplets, particularly relatively small droplets (for example, one micron in diameter), from aggregating into larger droplets which may result in settling (materials settling to the bottom) or creaming (oils rising to the top). For guideline purposes, it is believed most applications will involve the use of the Ostwald-ripening inhibitor in an amount of about 0.001 to about 5 wt. % of the composition and more likely in an amount of about 0.1 to about 1 wt. % of the composition.

The emulsification of the present composition may be effected through the use of one or more suitable surfactants. The selection of a suitable surfactant is deemed to be within the scope of those skilled in the art based on the teachings herein. Such surfactants include for example, anionic, cationic, and non-ionic surfactants. Preferred surfactants are non-ionic surfactants. Alone or in combination with one or more other surfactants, those having a hydrophilic-lipophilic balance number (HLB) of from about 4 to about 18 are preferred, those between 7 and 14 more preferred, and those between 9 and 13 most preferred. Examples of such non-ionic surfactants are PEG-60 corn glycerides, PEG-20 sorbitan monostearate, phenoxy-poly(ethyleneoxy)ethanol, sorbitan monooleate, and the like. Especially preferred are compendial surfactants such as those described in compendia such as the Food Chemicals Codex, National Formulary, U.S. Pharmacopeia, and the Code of Federal Regulations. It is preferred that the average diameter of the droplets of the emulsion be from about 50 nanometers (nm) to about 20 micrometers (μm) and more preferably from about 200 nm to about 5 μm. In general each surfactant is present in an amount no greater than about 2 wt. % of the composition and more generally no greater than about 1 wt. % of the composition. Also, it is important to prefer the nature of the side-chains of the surfactants to those with no double bonds, and this invention is most preferred to include those without unsaturated carbon-carbon bonds. The reason for this is that unsaturated fatty acid side chains (called also “olefinic” fatty acids) tend to oxidize over time, rendering them unsuitable. They tend to become colored, or dark, and give rise to intermediates that may react with the important antigen in the same formulation, rendering it less useful or unsuitable from a regulatory vantage point (in the US, for example, the key regulatory body being the FDA, and in other countries its counterpart). Olefins are suspected to have the additional liability of contributing to irritation which must be avoided for intranasal applications. However, unsaturated side-chain surfactants are not excluded from use in this invention. For example, polysorbate 80, containing a monounsaturated side chain of oleic acid ester, may be mitigated in its irritation liability by using a limited concentration of same, generally under 1% in the formulation, or by adding soothing components, such as glycerin, to the formulation to negate such undesired effect.

In some instances the permeation enhancers, benzodiazepine and/or other components of the compositions used in the instant invention may crystallize at room temperature or at higher temperatures. In order to inhibit or prevent such crystallization, the composition may include one or more crystallization inhibitors. Crystallization, if allowed to proceed, renders the emulsion unstable and has an adverse effect on shelf life. Preferred crystallization inhibitors function by lowering the temperature at which the involved agent crystallizes. Examples of such crystallization inhibitors include natural oils, oily substances, waxes, esters, and hydrocarbons. Examples of natural oils or oily substances include Vitamin E acetate, octyl palmitate, sesame oil, soybean oil, safflower oil, avocado oil, palm oil, and cottonseed oil. Another example of such a crystallization inhibitor is polyethylene glycol 1000. The selection of a suitable crystallization inhibitor is deemed to be within the scope of those skilled in the art from the teachings herein.

For example, crystallization inhibitors may be capable of lowering the temperature of crystallization of the involved agent to below about 25° C., or to below about 5° C. Examples of especially preferred crystallization inhibitors for use in inhibiting the crystallization of oxacyclohexadecan-2-one include hexadecane, isopropyl myristate, octyl palmitate, cottonseed oil, safflower oil, and Vitamin E acetate, each of which may be used in pharmaceutical preparations.

The crystallization inhibitor is present in the composition in a concentration effective to inhibit the crystallization of at least one component of the composition. In general the crystallization inhibitor is present in an amount of about 0.001 to about 5 wt. %, or from about 0.01 to about 2 wt % of the composition. In one embodiment, the crystallization inhibitor is present in an amount of from about 0.1 to about 1 wt. % of the composition. For example, a crystallization inhibitor is preferably used when the enhancer has a crystallization temperature above about 0 degrees Centigrade. In particular, for example, a crystallization inhibitor is preferably used when the enhancer is, pentadecalactone and/or cyclohexadecanone, since these crystallize above room temperature.

### EXAMPLES

The Examples below are illustrative of compositions of the present invention and are not to be construed as limiting.

### Example 1 In Vivo Study of Intranasal Diazepam Formulations

### Study I

This study's objective was to evaluate and characterize the pharmacokinetic and pharmacodynamic effectiveness of diazepam formulations after intranasal delivery to Yucatan minipigs. This study was performed in accordance with the NIH “Guide for the Care and Use of Laboratory Animals” and the federal Animal Welfare Act, and followed a protocol approved by the University of New Hampshire Institutional Animal Care and Use Committee.

### Formulations

Four diazepam formulations of the present invention (Formulations A, B, C and D) for use in intranasal sprays were prepared according to Table 1 below. Component CPE-215 is also known as cyclopentadecanolide or oxacyclohexadecan-2-one.

TABLE 1

Formulation

| | A | B | C | D |
|---|---|---|---|---|
| Reagent | % w/w | % w/w | % w/w | % w/w |
| Diazepam | 3 | 10 | 4 | 2.5 |
| Ethanol 200 proof | 10 | — | — | 10 |
| CPE-215 | 20 | 30 | 4 | 5 |
| Cremophor EL or | 10 | — | — | — |
| any forms of | | | | |
| polyethoxylated | | | | |
| castor oil | | | | |
| Dipropylene Glycol | 37 | 16 | 82 | — |
| Dimethyl Isosorbide | — | 44 | — | — |
| Propylene Glycol | — | — | — | 81 |
| H₂O | 20 | — | 10 | 1 |
| Total (% w/w) | 100 | 100 | 100 | 100 |

Three of these formulations (B, C, and D) were dispensed from intranasal atomizers, developed for humans by Valois of America. One puff of 100 microliters was instilled to pigs that had previously been cannulated with an indwelling jugular catheter. An extension to the actuator was used, provided also by Valois of America, to deliver formulation to the absorptive surface of the vestibule and labyrinthine turbinate region. The same actuator, dispensing 100 microliters, was used with the extension attached.

These intranasally delivered doses were compared to Diastat® (diazepam rectal gel) as a positive control.

### Animal Protocol

Female Yucatan miniature swine (pigs) were purchased from the UNH Miniature Swine Research Farm. During the study, the pigs were housed in an environmentally controlled research animal room (temperature 25+/−2 C and 12 hour light/dark cycle), fed commercial research pig chow, and had free access to water at all times. The pigs were 21 week-old Yucatan females weighing about 16-22 kg.

### Catheterization Methodology

The animals were prepared for the study with the surgical implantation of a jugular catheter 4 to 6 days before the study start. After heavy sedation with an intramuscular dose of xylazine and ketamine, the animals were masked down and maintained to effect deep surgical anesthesia with inhalation of isofluorane anesthetic and oxygen. With the animals in dorsal recumbency, a skin incision was made in the right jugular furrow and followed by blunt dissection of the subcutaneous and perivascular connective tissues to expose the right jugular vein cranial to the thoracic inlet. A length of 0.050 inch bore Tygon® intravenous catheter tubing was inserted through a small incision in the clamped vein and positioned caudally into the anterior vena cava (approximately 12 to 15 cm from the jugular incision). The catheter was transfixed to the vein and deep subcutaneous tissues suture. The jugular vein cranial to the catheter was ligated with polypropylene suture. The free end of the catheter was routed by blunt dissection through the subcutis to the interscapular dorsum, pulled through a small skin incision, and transfixed to the skin with polypropylene suture. The catheter was capped with a syringe docking device and filled with an anti-thrombotic preparation. The anti-thrombotic consisted of 60% (w/v) polyvinylpyrrolidone (10,000 mw, PVP-10) and physiologic saline/sodium heparin (50,000 units of heparin per 100 mL). The subcutis and skin incision in the jugular furrow were closed with synthetic absorbable and polypropylene sutures, respectively.

Diazepam was dosed either intravenously in the ear vein with a 22 gauge needle, rectally with a syringe, or sprayed into the nose of the animals (100 μL volume sprayed in one nostril).

The animals were comfortably bandaged to protect the catheter and skin incision and covered with a vest made for dog catheter work. Intramuscular butorphanol was administered as an analgesic immediately and 12 hours after surgery.

### Study Design

At the time of dosing, the pigs were restrained in a cloth sling. The pigs were afterwards free to move about their respective individual pens and were only temporarily restrained in close quarters at the front of the pen with a movable wooden gate at the time of blood sampling. Each pig was dosed twice with each of the formulations over a two week period with at least 18 hours between treatments. The intranasal dosing (Formulations B, C, D) entailed dispensing 100 μL of a diazepam formulation through an aerosol doser (human type intranasal actuator), into one nostril. For intranasal dosing, it is important to hold the nose up for at least 5 seconds after the dosing to make sure no formulation will drip out later. Dosing interval between pigs was timed, and was approximately five minutes.

For rectal dosing, 0.8 mL of 0.5% Diastat rectal gel (4 mg dose) was administered to each pig which was fastened on the sling. 1-ml syringe coated with viscous PVP solution was used as lubricant.

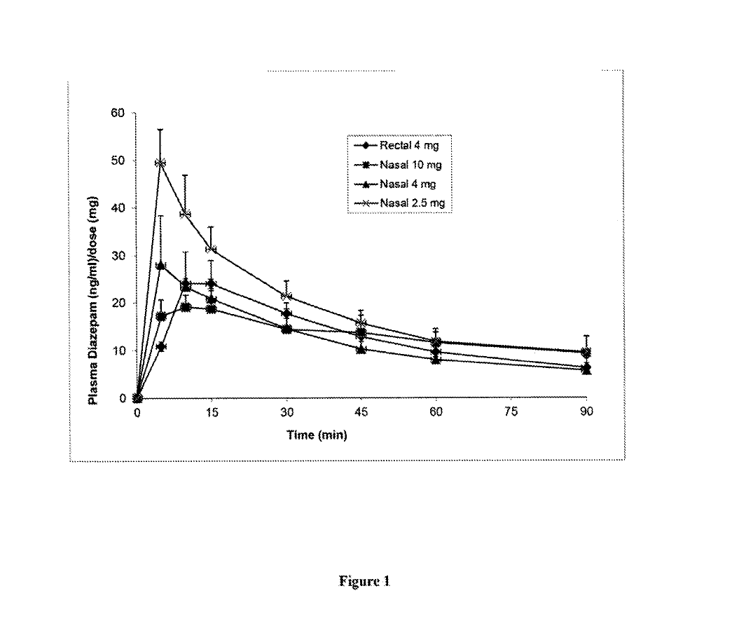

For the study in FIG. 1, pigs were treated according to the schedule below (Table 2): Diastat (n=6): Diastat rectal gel, 0.5% diazepam, 0.8 mL (4 mg dose)

Formulation B (n=6): 10% nasal diazepam formulation.
Formulation C (n=6): 4% nasal diazepam formulation.
Formulation D (n=6): 2.5% nasal diazepam formulation.

TABLE 2

| | Pig 1 | Pig 2 | Pig 3 | Pig 4 |
|---|---|---|---|---|
| Week #1 | | | | |
| Day 1 (Mon) | C | D | C | D |
| Day 2 (Tue) | | | | |
| Day 3 (Thu) | Diastat | Diastat | B | B |
| Day 4 (Fri) | | | | |
| Week #2 | | | | |
| Day 5 (Mon) | | | | |
| Day 6 (Tue) | D | C | D | C |
| Day 7 (Wed) | | | | |
| Day 8 (Fri) | B | B | B | B |
| Week #3 | | | | |
| Day 9 (Mon) | | | | |
| Day 10 (Tue) | C | D | C | D |
| Day 11 (Thu) | | | | |
| Day 12 (Fri) | Diastat | Diastat | Diastat | Diastat |

Baseline venous blood specimens were collected just prior to intranasal or rectal diazepam administration, and 2 ml blood was thereafter sampled at 0 (just before treatment), 5, 10, 15, 30, 45, 60 and minutes after application, or was sampled at 0, 10, 20, 30, 40, 50, 60 and 90 minutes after application. Bleeding was approximately five minutes apart between pigs, the interval adjusted relative to the dosing time within one minute of target time.

The blood was collected into sodium heparinized glass tubes. EDTA plasma samples were retrieved and assayed immediately or stored frozen at −20° C. or below until analyzed for diazepam. Repeated freezing and thawing of specimens should be avoided.

### Sample Collection and Assay Methods

Heparinized plasma was analyzed for diazepam concentration using the LC/MS/MS methods. Briefly, sample preparation was by protein precipitation using a deuterated internal standard, Diazepam-D₅. Prior to sample analysis, a method performance assessment was conducted with evaluations of specificity, matrix effects, calibration curve, LLOQ, carryover, precision and accuracy. Sample analysis was conducted over two days with samples from each pig run with duplicate QCs at three levels and bracketing calibration curves. Experimental procedures are detailed below.

A 1 mg/mL Diazepam certified reference solution in methanol (Catalog No. D-907) was purchased from Cerilliant (Round Rock, Tex.). A 100 μg/mL Diazepam-D₅ internal standard certified reference solution in methanol (Catalog No. D-902) was also purchased from Cerilliant. Yucatan miniature swine blank plasma with K3EDTA anticoagulant was purchased from Bioreclamation, including 3 male lots and 3 female lots. Lots were screened individually for interference and then pooled for the preparation of calibration standards and QCs. ZORBAX Eclipse XDB-C18 HPLC column was purchased from Agilent (971700-902, 2.1×50 mm, 3.5-μm particle size). AB Sciex API 4000 mass spectrometer with Analyst 1.4.2 software was used.

Working solutions were prepared in methanol from the 1.000 mg/mL Diazepam stock solution at the following concentrations: 100000, 60000, 40000 and 20000 ng/mL. Each of these solutions was diluted 20-fold in blank plasma resulting in concentrations of 5000, 3000, 2000, and 1000 ng/mL. Calibration standards and quality control (QC) samples were prepared in blank plasma by serial dilution of the 5000, 3000, 2000, and 1000 ng/mL Diazepam prepared in plasma. Standards were prepared at the following nominal concentrations: 500, 400, 200, 100, 50, 20, 10, 5, 2 and 1 ng/mL. QC samples were prepared at the following nominal concentrations: 300, 30 and 3 ng/mL.

A batch comprised specimens from a single pig, duplicate QCs at three levels placed after approximately one-third and two-thirds of the specimens, and bracketing ten-point calibration curves.

Plasma (50 μL) was added to a 96-well plate. Acetonitrile containing 50 μg/mL of Diazepam-D₅ (150 μL) was added. The plate was placed on an orbital shaker at 160 rpm for 10 minutes at ambient temperature and then centrifuged at 4000 rpm for 10 min at ambient temperature. An aliquot of supernatant was removed (50 μL), transferred to another 96-well plate, and diluted with 50 μL of mobile phase A (0.1% formic acid).

Chromatographic parameters were as follows: column used was ZORBAX Eclipse XDB-C18, 2.1×50 mm, 3.5 μm; mobile phase A was 0.1% formic acid in water; mobile phase B was 0.1% formic acid in ACN; column temperature was 45° C. Gradient (linear) was: 0.00-2.50 min, 30-100% B; 2.50-3.00 min, 100% B; 3.00-3.10 min, 100-30% B; 3.10-4.60 min, 30% B (with additional re-equilibration time from autosampler rinse cycle). Injection volume was 10 μL. Autosampler wash was 0.1% formic acid in ACN. Retention time for Diazepam and Diazepam-D₅ was 2.1 min and 2.07 min, respectively.

Mass spectrometer parameters were as follows: ionization mode was electrospray (TurbolonSpray), positive ion; gas 1 (GS1) was 65; gas 2 (GS2) was 65; curtain gas (CUR) was 20; IonSpray voltage (IS) was 4000; source temperature (TEM) was 400° C.; declustering potential (DP) was 70; entrance potential (EP) was 9; acquisition mode was SRM. Diazepam transition was: m/z 285.1-193.0; dwell=75 msec; collision energy (CE)=46. Diazepam-D₅ transition was: m/z 290.1-198.0; dwell=75 msec; collision energy (CE)=46. Divert valve divert to waste at 2.50 min and back to MS at 4.5 min.

### CONCLUSIONS

Intranasal formulations B, C and D can rapidly and effectively deliver diazepam systemically to the Yucatan pig. The intranasal formulations could provide similar or much higher diazepam bioavailability compared to rectal formulation Diastat (Table 3).

TABLE 3
Relative Bioavailability
Relative Bioavailability

| DiaStat | Formulation B | Formulation C | Formulation D |
|---|---|---|---|
| (rectal, 4 mg) | (nasal, 10 mg) | (nasal, 4 mg) | (nasal, 2.5 mg) |
| 100% | 101% | 93% | 146% |

Tmax of the intranasal formulations ranged from about 5 to about 10 minutes, while Tmax of the rectal formulation Diastat was about 15 minutes (FIG. 1). In FIG. 1, the dose-normalized Cmax of plasma benzodiazepine after administration of the present compositions was about 50 ng/ml/mg for the 2.5 mg dose diazepam composition, about 27.5 ng/ml/mg for the 4 mg dose diazepam composition, and about 18 ng/ml/mg for the 10 mg dose diazepam composition. Nasal formulations were more reproducible (having less variations) than rectal Diastat.

The AUC values and dose-normalized AUC values for Formulations B, C and D are shown in Tables 4 and 5, respectively.

TABLE 4
AUC

| | AUC₀₋ₜ (ng*min/ml) | | | |
|---|---|---|---|---|
| t | DiaStat | Formulation B | Formulation C | Formulation D |
| (min) | (rectal, 4 mg) | (nasal, 10 mg) | (nasal, 4 mg) | (nasal, 2.5 mg) |
| 0 | 0 | 0 | 0 | 0 |
| 5 | 108.3 | 429 | 281.05 | 309.3 |
| 10 | 458.2 | 1335 | 796.1 | 860.3 |
| 15 | 940.4 | 2278.5 | 1238.7 | 1297.9 |
| 30 | 2192.9 | 4761 | 2300.8 | 2285 |
| 45 | 3108.7 | 6876.3 | 3044.7 | 2979 |
| 60 | 3781 | 8776.5 | 3590.2 | 3495.4 |
| 90 | 4723.8 | 11911.8 | 4402 | 4296.1 |

TABLE 5
Dose-normalized AUC

| | AUC₀₋ₜ (ng*min/ml/mg) | | | |
|---|---|---|---|---|
| t | DiaStat | Formulation B | Formulation C | Formulation D |
| (min) | (rectal, 4 mg) | (nasal, 10 mg) | (nasal, 4 mg) | (nasal, 2.5 mg) |
| 0 | 0 | 0 | 0 | 0 |
| 5 | 27.8 | 42.9 | 70.3 | 123.7 |
| 10 | 114.6 | 133.5 | 199 | 344.1 |
| 15 | 235.1 | 227.9 | 309.7 | 519.2 |
| 30 | 548.2 | 476.1 | 575.2 | 914 |
| 45 | 777.2 | 687.6 | 761.2 | 1191.6 |
| 60 | 945.2 | 877.7 | 897.6 | 1398.2 |
| 90 | 1181 | 1191.2 | 1100.5 | 1718.4 |

### Example 2 In Vivo Study of Intranasal Diazepam Formulations

### Study H

### Formulations

Three diazepam formulations of the present invention (Formulations E, F and G) for use in intranasal sprays were prepared according to Table 6 below.

TABLE 6

| | Formulation | | |
|---|---|---|---|
| | E | F | G |
| Reagent | % w/w | % w/w | % w/w |
| Diazepam | 2 | 1 | 2 |
| Ethanol 200 proof | 10 | 10 | — |
| CPE-215 | 5 | 5 | — |
| Propylene Glycol | 82 | 83 | 98 |
| H₂O | 1 | 1 | — |
| Total (% w/w) | 100 | 100 | 100 |

The above formulations in Table 6 were able to dissolve diazepam up to 25 mg/ml. Higher concentrations of diazepam may also be achieved by the present invention. An evaluation of Formulations E, F and G was carried out in vivo according to the animal protocol as described in Example 1.

### CONCLUSIONS

Intranasal formulations E, F and G can rapidly and effectively deliver diazepam systemically to the Yucatan pig. The intranasal formulations could provide much higher diazepam bioavailability compared to rectal formulation Diastat (Table 7).

TABLE 7
Relative Bioavailability
Relative Bioavailability

| DiaStat | Formulation E | Formulation F | Formulation G |
|---|---|---|---|
| (rectal, | (nasal, 2 mg, | (nasal, 1 mg, | (nasal, 2 mg, |
| 4 mg) | CPE-215) | CPE-215) | propylene glycol) |
| 100% | 281% | 293% | 262% |

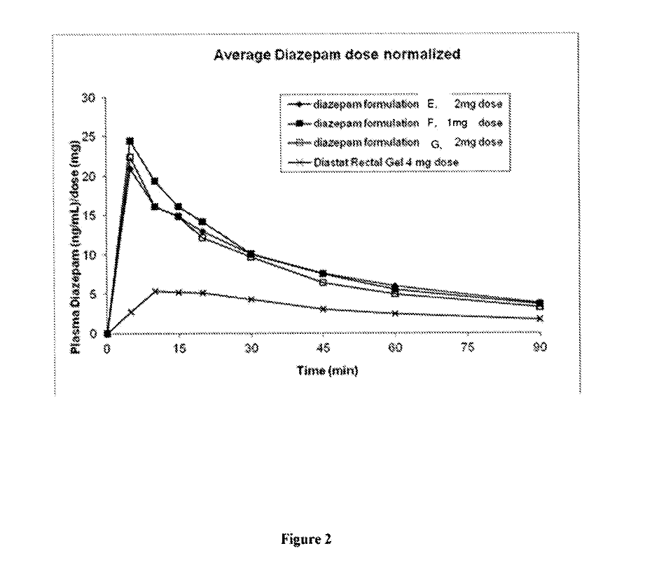

Tmax of the intranasal formulations ranged from about 5 to about 10 minutes, while Tmax of the rectal formulation Diastat was about 10 to 15 minutes (FIG. 2). In FIG. 2, Cmax of plasma diazepam was about 40-45 ng/ml for the 2 mg dose diazepam composition with or without CPE-215, and about 23 ng/ml for the 1 mg dose diazepam composition. Nasal formulations were more reproducible (having less variations) than rectal Diastat.

The AUC values and dose-normalized AUC values for Formulations E, F and G are shown in Tables 8 and 9, respectively.

TABLE 8
AUC

| | AUC₀₋ₜ (ng*min/ml) | | | |
|---|---|---|---|---|
| | DiaStat | Formulation E | Formulation F | Formulation G |
| t | (rectal, | (nasal, 2 mg, | (nasal, 1 mg, | (nasal, 2 mg, |
| (min) | 4 mg) | CPE-215) | CPE-215) | propylene glycol) |
| 0 | 0 | 0 | 0 | 0 |
| 5 | 26.9 | 104.6 | 60.9 | 112.1 |
| 10 | 107.1 | 289.9 | 170.2 | 304.5 |
| 15 | 212 | 444.8 | 258.7 | 458.9 |
| 20 | 314.7 | 583.5 | 334.3 | 593.6 |
| 30 | 500.5 | 812.5 | 455.2 | 811.6 |
| 45 | 715.3 | 1075.7 | 586.4 | 1051.9 |
| 60 | 876.2 | 1248.4 | 683.9 | 1220.1 |
| 90 | 1117.6 | 1568.6 | 820 | 1461.6 |

TABLE 9
Dose-normalized AUC

| | AUC₀₋ₜ (ng*min/ml/mg) | | | |
|---|---|---|---|---|
| | DiaStat | Formulation E | Formulation F | Formulation G |
| t | (rectal, | (nasal, 2 mg, | (nasal, 1 mg, | (nasal, 2 mg, |
| (min) | 4 mg) | CPE-215) | CPE-215) | propylene glycol) |
| 0 | 0 | 0 | 0 | 0 |
| 5 | 6.7 | 52.3 | 60.9 | 56 |
| 10 | 26.8 | 145 | 170.2 | 152.3 |
| 15 | 53 | 222.4 | 258.7 | 229.5 |
| 20 | 78.7 | 291.8 | 334.3 | 286.8 |
| 30 | 125 | 406.3 | 455.2 | 405.8 |
| 45 | 178.8 | 537.9 | 586.4 | 526 |
| 60 | 219 | 624.2 | 683.9 | 610 |
| 90 | 279.4 | 784.3 | 820 | 730.8 |

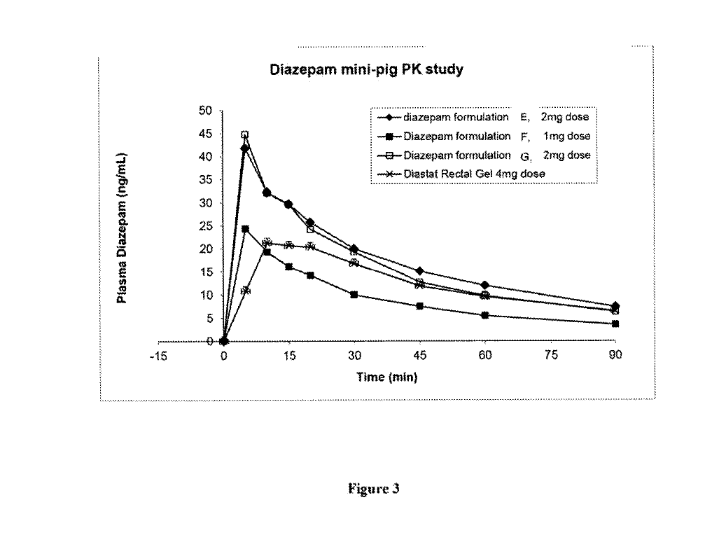

FIG. 3 shows that when the data is presented without dose normalization, there was dose linearity when comparing Formulations E (2% diazepam) to F (1% diazepam). The AUC for Formulation E was 1.9 fold that of Formulation F. Formulation G had the same AUC as Formulation E, showing that propylene glycol is a good solvent for diazepam.

### Example 3 In Vivo Study of Rectal Diazepam Formulation Diastat and Intravenous Diazepam Formulation

21-week-old healthy female Yucatan miniature pigs, weighing about 16-22 kg, were dosed diazepam either rectally with 4 mg of diazepam in diastat (2 animals; 0.8 ml of 0.5% diazepam in diastat), or intravenously in ear vein with a 22 gauge needle (4 animals) with 2 mg diazepam dissolved at 0.5% in 0.4 nil of 15% ethanol, 40% propylene glycol, 43% water and 1.5% benzyl alcohol.

Blood (2 ml) was collected before dosing (time 0), and at 5, 10, 15, 30, 45, and 60 minutes post dosing and analyzed for diazepam.

### Results

The areas under the curves (AUC) were calculated, and the bioavailability of rectally administered diazepam was determined by calculating the ratio of rectally/intravenous (IV) AUCs normalized for dose. The bioavailability of rectally administered diazepam in Diastat was found to be 57%.

### Example 4 Stability Study of Diazepam Formulations

The stability of diazepam in the formulations described above (Formulations A-D in Table 1) was assessed in viol). Aliquots of Formulations A-D were stored at room temperature (about 25° C.) or 40° C. The concentration of diazepam was then analyzed after 0, 1, 2 and 3 months of storage. The concentrations of diazepam in the formulations were determined by HPLC using a Zorbax C18, 5 um, 150×4.6 mm column. The column running conditions are: column temp: 40° C., run time: 12 min, flow rate: 1.25 mL/min, and mobile phase: methanol: water (60:40). Diazepam was detected by UV absorption. The concentration was determined from a calibration graph of 30 to 300 μg/mL diazepam, and shown in Tables 10 and 11.

In all four formulations tested, diazepam was fairly stable after 3 months of storage at both 25° C. and 40° C. There was no or little precipitation or break down of diazepam observed. Note, in Tables 10 and 11, Lot #88A, 88B, 90A and 90B are Formulations A, B, C and D in Table 1, respectively.

TABLE 10
Stability of Diazepam Formulations at 25° C.

| | | Time | Time | Time | Time |
|---|---|---|---|---|---|
| | | Point | Point | Point | Point |
| Lot # | Parameter | (Initial) | (1 Month) | (2 Months) | (3 Months) |
| 88A | Diazepam | 3.01 | 3.33 | 2.67 | 2.99 |
| 88B | content | 9.03 | 10.93 | 8.68 | 9.08 |
| 90A | (%) | 4.06 | 4.49 | 3.53 | 3.16 |
| 90B | | 2.51 | 2.76 | 2.22 | 2.20 |

TABLE 11
Stability of Diazepam Formulations at 40° C.

| | | Time Point | Time Point | Time Point | Time Point | Tune Point | Time Point |
|---|---|---|---|---|---|---|---|
| Lot # | Parameter | (Initial) | (Week 2) | (1 Month) | (Week 6) | (2 Months) | (3 Months) |
| 88A | Diazepam | 3.01 | 3.02 | 3.36 | 3.32 | 3.01 | 2.90 |
| 88B | content (%) | 9.03 | 10.21 | 9.08 | 10.92 | 8.71 | 8.96 |
| 90A | | 4.06 | 4.17 | 4.46 | 4.35 | 3.52 | 3.87 |
| 90B | | 2.51 | 2.63 | 2.72 | 2.75 | 2.21 | 2.48 |

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

All patents, applications, publications, test methods, literature, and other materials cited herein are hereby incorporated by reference.

What is claimed is:

1. A method for treating a disorder or a disease, comprising administering intranasally to a nasal mucosa a pharmaceutical composition for nasal administration including a therapeutically effective amount of diazepam and oxacyclohexadecan-2-one in a concentration effective to enhance the penetration of the diazepam through the nasal mucosa.

2. The method of claim 1, wherein, after administration of a single dose of the pharmaceutical composition, Tₘₐₓ of plasma diazepam ranges from about 1 minute to about 10 minutes, and Cₘₐₓ of plasma diazepam ranges from about 15 to about 800 ng/ml when plasma diazepam is measured from about 0 to about 2 hours.

3. The method of claim 1, wherein dose-normalized Cₘₐₓ of plasma diazepam ranges from about 3 to about 60 ng/ml/mg.

4. The method of claim 1, wherein AUC_{0-5 min} of plasma diazepam ranges from about 50 to about 500 ng*min/ml.

5. The method of claim 1, wherein AUC₀₋₁₀ mm of plasma diazepam ranges from about 150 to about 1,400 ng*min/ml.

6. The method of claim 1, wherein the composition is substantially non-irritating to the nasal mucosa.

7. The method of claim 1, wherein the amount of the diazepam ranges from about 0.1% (w/w) to about 50% (w/w) of the composition.

8. The method of claim 7, wherein the amount of the diazepam ranges from about 1% (w/w) to about 10% (w/w) of the composition.

9. The method of claim 1, wherein the oxacyclohexadecan-2-one ranges from about 0.001% (w/w) to about 40% (w/w) of the composition.

10. The method of claim 9, wherein the oxacyclohexadecan-2-one ranges from about 1% (w/w) to about 10% (w/w) of the composition.

11. The method of claim 1, wherein the disorder or the disease is selected from anxiety, epilepsy, insomnia, agitation, seizures, muscular disorders, alcohol dependence, and drug withdrawal.
